(19) United States
(12) Patent Application Publication
(10) Pub. No.: US 2025/0234975 A1
(43) Pub. Date: Jul. 24, 2025

(54) ARRANGEMENT APPLIED TO A STEAM DEVICE FOR CLEANING AND SANITISING THE BLADES OF SHAVING APPARATUS

(71) Applicant: Gilberto Américo Just de NOVAES, RECIFE (BR)

(72) Inventor: Gilberto Américo Just de NOVAES, RECIFE (BR)

(21) Appl. No.: 18/699,798

(22) PCT Filed: Jul. 8, 2022
(86) PCT No.: PCT/BR2022/050253

§ 371 Date: Apr. 9, 2024

(51) Int. Cl. A45D 27/46; A61L 2/07; A61L 2/24

(52) U.S. Cl. CPC A45D 27/46; A61L 2/07; A61L 2/24; A61L 2202/11; A61L 2202/122; A61L 2202/14; A61L 2202/15

(57) ABSTRACT

Autonomous and compact, formed by a partitioned cabinet composed of main body (3) where an L-shaped compartment (7) acts as water reservoir, utilized to clean and sanitize the blades (2) of razors (13) during use, by the positioning on a support (14) in the central compartment (8) which is part of a drive mechanism (M) of an electric pump (12), which pressurizes the water towards a microboiler (30) making the steam (V) at high pressure and temperature the protagonist of the cleaning.

### INTRODUCTION

The present application for utility model patent refers to an unprecedented device that cleans and sanitizes razor blades in an efficient manner, utilizing steam at high pressure and temperature as protagonist, followed by washing with water, promoting the removal of hair and shaving cream of the blades per se.

### FIELD OF APPLICATION

The innovation is applied in the personal grooming sector, more specifically in the sector of shaving accessories.

### CONVICTION

During the act of shaving, the blades become dirty with hairs and shaving cream. It is usual to clean the disposable razor manually with running water, from faucets or from showers during bathing, as well as in mechanical contact between the blades and the tubs of the sinks. Either the procedures described above do not provide suitable cleaning or they are not hygienic. These procedures may also entail damage to the razors, causing shaving failures or cuts to the skin. The mechanical contact, remnants of hairs and shaving cream residue on the blades may reduce the useful life of the device, and also cause allergies and skin irritations.

In this context, new technologies that enable more efficient cleaning and facility for the user of the razor are welcome in the sector in question.

### STATE OF THE ART

The current state of the art anticipates certain patent documents that refer to devices for cleaning and sanitizing portable razor blades without mechanical contact, such as WO 15162582 A1 entitled “DEVICE FOR CLEANING A WET SHAVING APPARATUS”—consisting of an accommodation and a rinsing unit to rinse the head of the shaving blade. According to the invention, a vibrating table is supplied inside the accommodation such that the vibrating table is suitable at least for accommodating the blade head. Additionally, a cleaning process can be carried out with water, water jets and/or air.

In the document above, the differentiating factors is the use of a vibrating table for removing the hairs and residue from the blades supported on same. The dependent claims claim the use of the vibration jointly with UV lamp; water and power autonomy; pulsating water flow; disinfectant and simultaneous air jet.

Document U.S. Pat. No. 20,171,87234 AA entitled “MULTI-BLADE RAZOR CARTRIDGE CLEANER”—a device that cleans between the shaving blades with the water jet to remove the hair and the soap stuck during each passage of the razor over the surface of the skin. It comprises the water tank connected to a pump that pressurizes clean water and leads it by hose at least to a spray nozzle under the blade to be cleaned, the apparatus of which is resting on a high cradle. Beneath the blade there is a cleaning chamber that funnels it towards an elimination tank. The blade is covered by a protective cap that prevents water from splashing outside the device during cleaning.

The device anticipated in the document above performs the cleaning by means of a flow of pressurized water towards the nozzles, which is pumped into a clean water tank that does not prove effective. Depending on the degree of grime, the water pressure is not sufficient to remove the residue completely. Said device bears other technical limitations such as not performing an aseptic cleaning of the blades. The horizontal cradle on which the razor is supported is also a drawback, being susceptible to soaking with the water coming from the spray nozzle. Even with the liquid-containing upper cap, it is impossible to prevent the moist mist generated by the spray nozzle from flowing out into the environment. Lastly, the cabinet is not partitioned, which besides not aggregating the operating components in an organized manner also exposes the electronics to the harmful action of moisture.

Document ES 1221966 UA entitled “DEVICE FOR THE USE OF A MANUAL SHAVE MACHINE”'-characterized by comprising:—a first tank (3) containing at least a liquid fluid;—a second reservoir configured to collect said liquid fluid after having been used to carry out the cleaning of a head of the manual razor;—a third tank the inner space of which accommodates a cleaning mouthpiece which is configured to clean the head of the manual razor when said head is inside the third tank;—a system of distribution of liquid fluid configured to transport the liquid fluid from the first reservoir to the cleaning mouthpiece through which the liquid fluid conducted towards the head of the manual razor leaves when the liquid fluid drive system is activated.

The device anticipated in the above document uses two tanks, clean water and residual water, the upper inlet to support the shaving blade which is cleaned by the action of the water flow towards the mouthpiece by means of the manual pump.

### OBJECTIVES OF THE INNOVATION

It is an objective of the present innovation to propose an arrangement applied in a steam device for cleaning and sanitizing shaving blades, without mechanical contact, which uses water steam at high pressure and temperature as protagonist for cleaning the blades in a fast and practical manner.

It is an objective of the present innovation to propose an arrangement applied in a steam device for cleaning and sanitizing shaving blades which at the same time it cleans and sanitizes, performs the aseptic cleaning of the blade due to the high temperature steam and pressure.

It is an objective of the present innovation to propose an arrangement applied in a steam device for cleaning and sanitizing shaving blades that operates with steam as protagonist, susceptible to be followed by a flow of pressurized water to complement the cleaning.

It is an objective of the present innovation to propose an arrangement applied in a steam device for cleaning and sanitizing shaving blades whose partitioned cabinet accommodates the operating components in an organized manner, and also maintains the necessary seal-tightness so that the water contained in the reservoir and the moisture damage the electronics and other mechanisms.

It is an objective of the present innovation to propose an arrangement applied in a steam device for cleaning and sanitizing shaving blades wherein the positioning of the razor head with the blades is enclosed in a cleaning chamber accessed through the hatch, thus preventing soaking, mist into the environment and, consequently, more comfort for the user.

It is an objective of the present innovation to propose an arrangement applied in a steam device for cleaning and sanitizing shaving blades having dimensions that enable portability and autonomous operation, with rechargeable batteries and water reservoir with sufficient capacity for the full shaving operation.

It is an objective of the present innovation to propose an arrangement applied in a steam device for cleaning and sanitizing shaving blades susceptible to aggregating other technologies, such as, for example, audio, communication with wireless technology, UV light.

It is an objective of the present innovation to propose an arrangement applied in a steam device for cleaning and sanitizing shaving blades with optimal cost-benefit ratio.

### SUMMARY OF THE INNOVATION

The ARRANGEMENT APPLIED IN A STEAM DEVICE FOR CLEANING AND SANITIZING RAZOR BLADES—based on the use of water steam as protagonist of the cleaning and sanitization of the razor blades, which are also aseptic, susceptible to having said cleaning complemented by a flow of pressurized water, once the steam has been turned off, the working time of which can be controlled by the user. The device is assembled in a cabinet partitioned whose body has an L-shaped compartment comprising a water seal-tight reservoir in order to avoid external and internal leakage, which prevents damage to the mechanical and electronic components such as the water-pressurizing pump installed in a regular prism-shaped compartment, which follows on to a microboiler, under the aforementioned body, and from there to an injector nozzle with different outlets that direct the steam and water to the blades of the razor in the central compartment, the residual water being drained to a lower compartment.

### DESCRIPTION OF THE DRAWINGS

In sequence, drawings are set forth for greater clarification of the patent application, in an illustrative, non-limitative manner:

FIG. 1: Perspective view of the arrangement applied in a steam device for cleaning and sanitizing shaving blades.

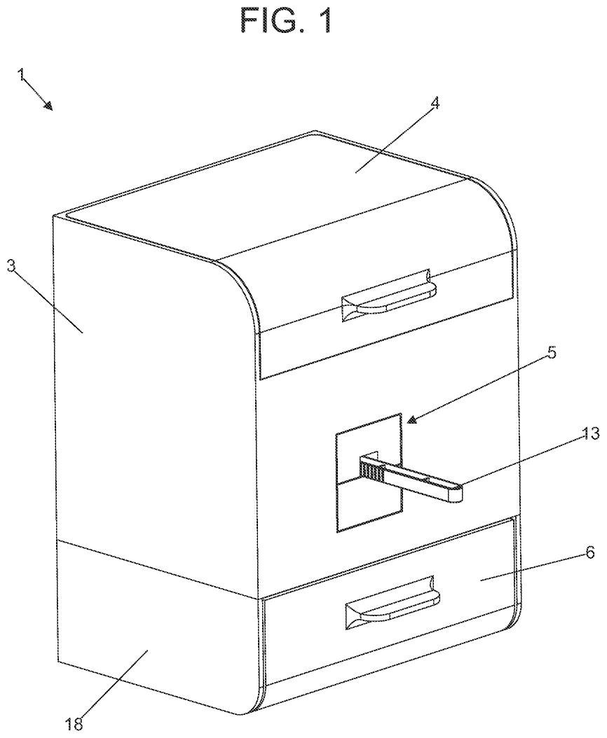

FIG. 2: Blown-up perspective view of the arrangement applied in a steam device for cleaning and sanitizing shaving blades.

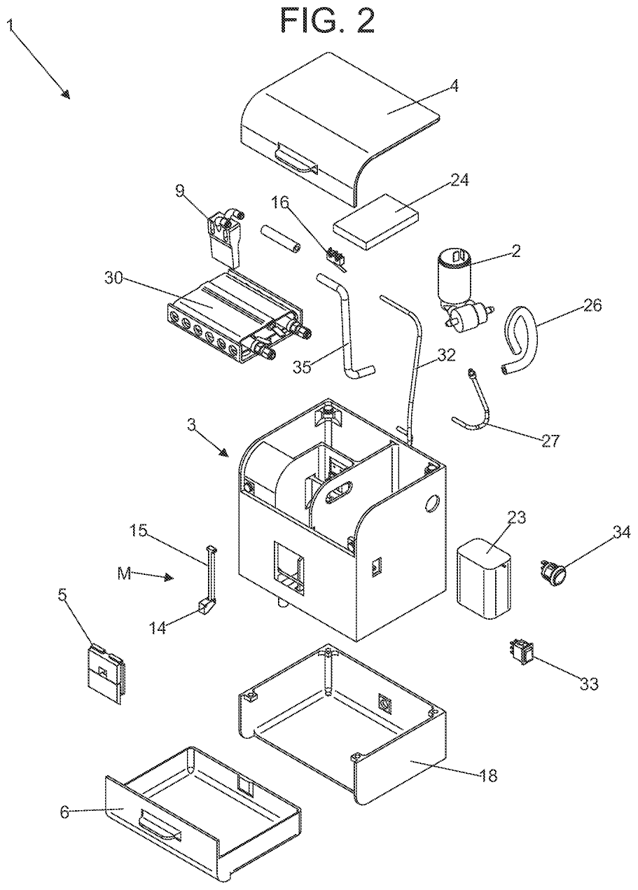

FIG. 3: Schematic diagram with the main components of the arrangement applied in a steam device for cleaning and sanitizing shaving blades.

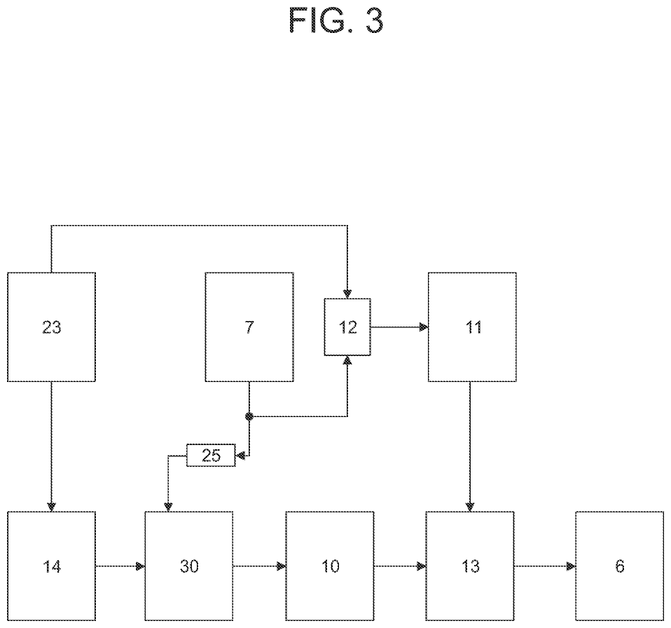

FIG. 4: Side cutaway view of the arrangement applied in a steam device for cleaning and sanitizing shaving blades.

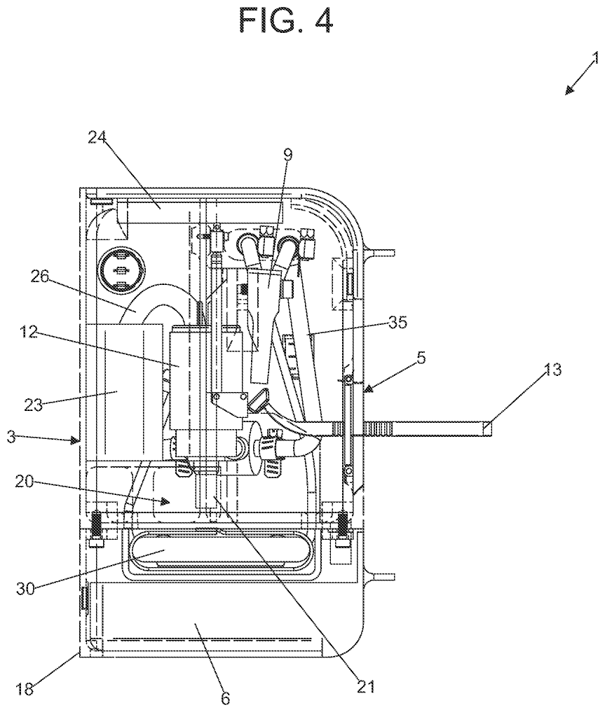

FIG. 5: Opposite side cutaway view of the arrangement applied in a steam device for cleaning and sanitizing shaving blades.

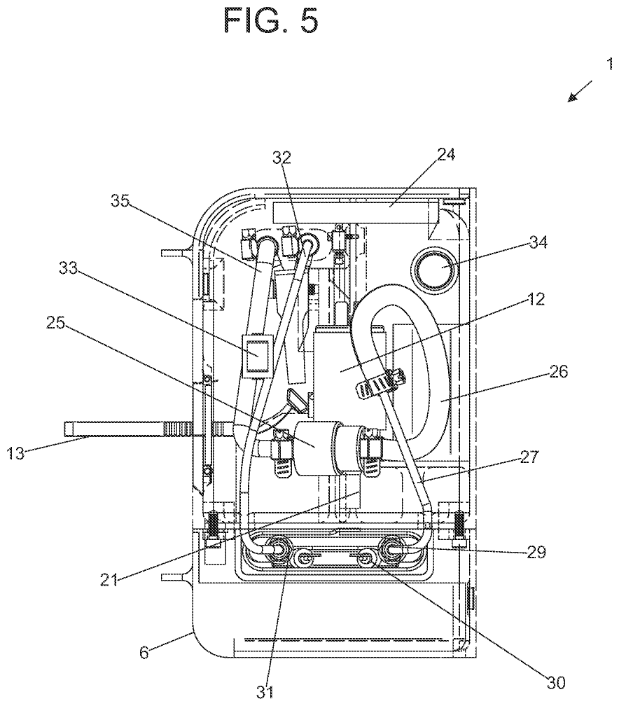

FIG. 6: Perspective view illustrating the hydraulic connections to the injector nozzle of the arrangement applied in a steam device for cleaning and sanitizing shaving blades.

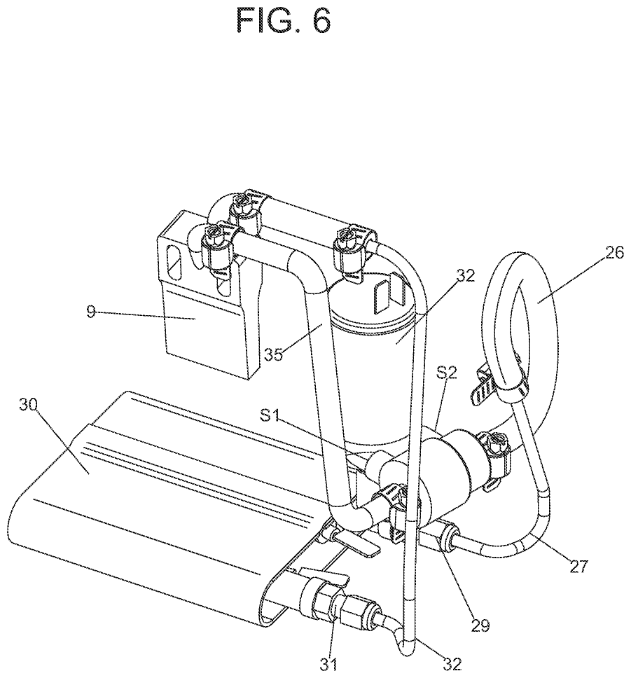

FIG. 7: Cutaway front view of the arrangement applied in a steam device for cleaning and sanitizing shaving blades.

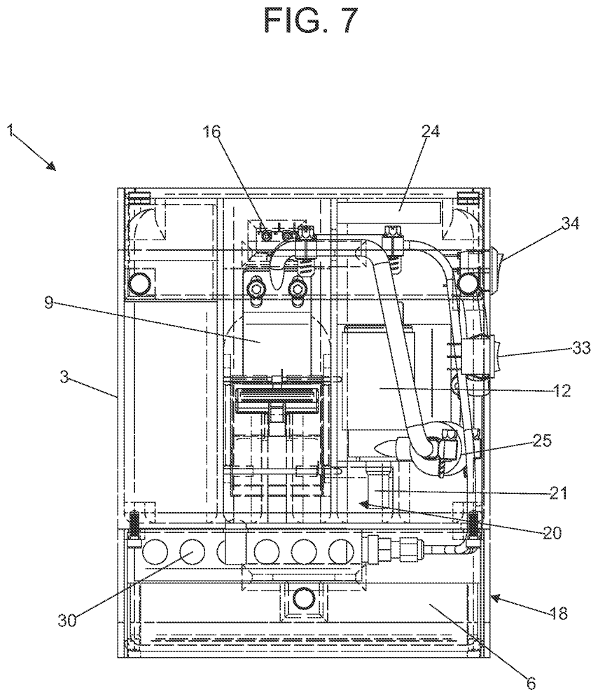

FIG. 8: Cutaway top view of the arrangement applied in a steam device for cleaning and sanitizing shaving blades.

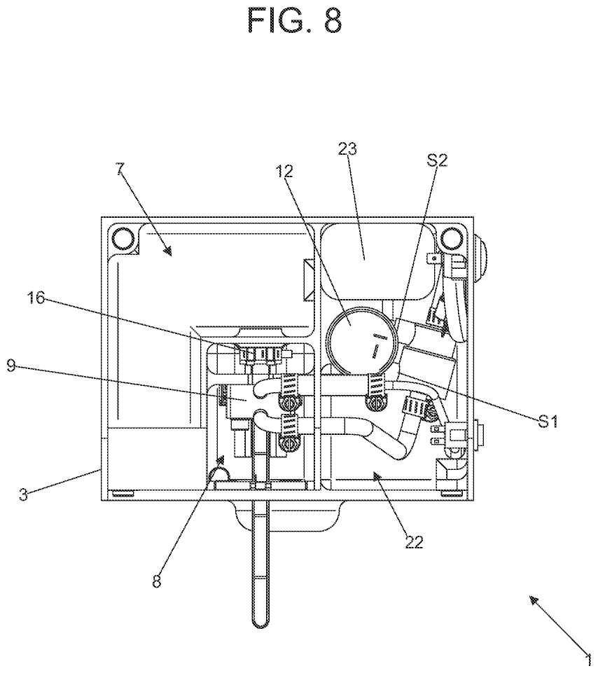

FIG. 9: Top perspective view of the arrangement applied in a steam device for cleaning and sanitizing shaving blades, without the cap.

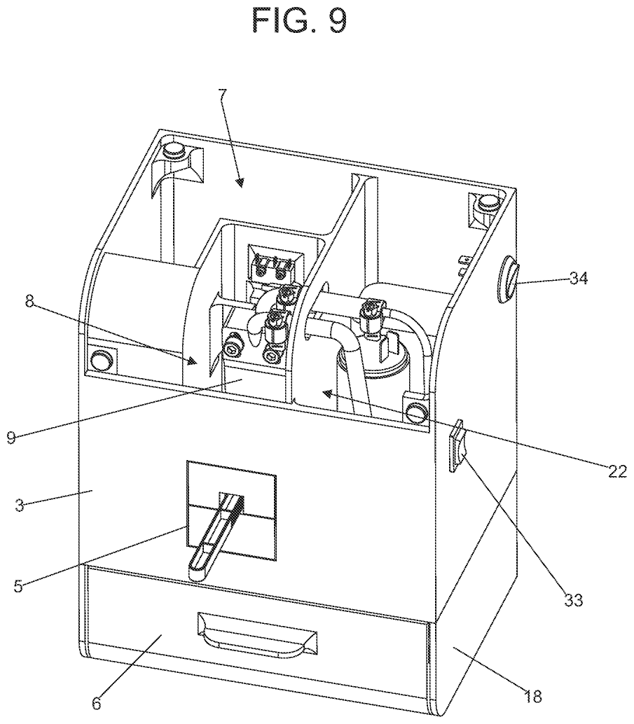

FIG. 10: Perspective view of the cabinet body of the arrangement applied in a steam device for cleaning and sanitizing shaving blades.

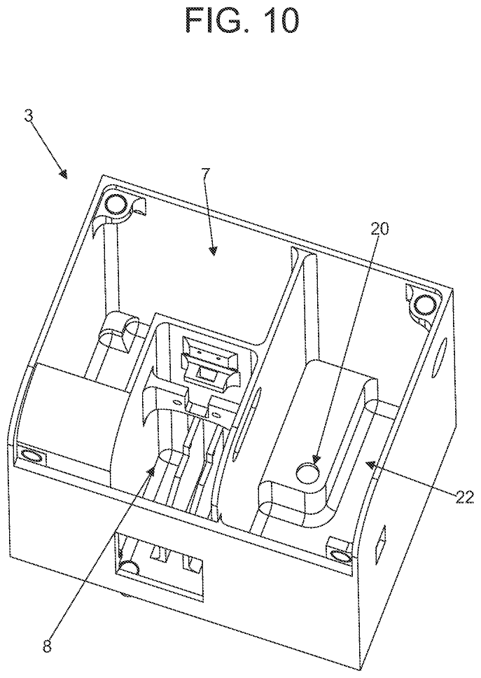

FIG. 11: Detail of the communication between the water reservoir and the pump of the arrangement applied in a steam device for cleaning and sanitizing shaving blades.

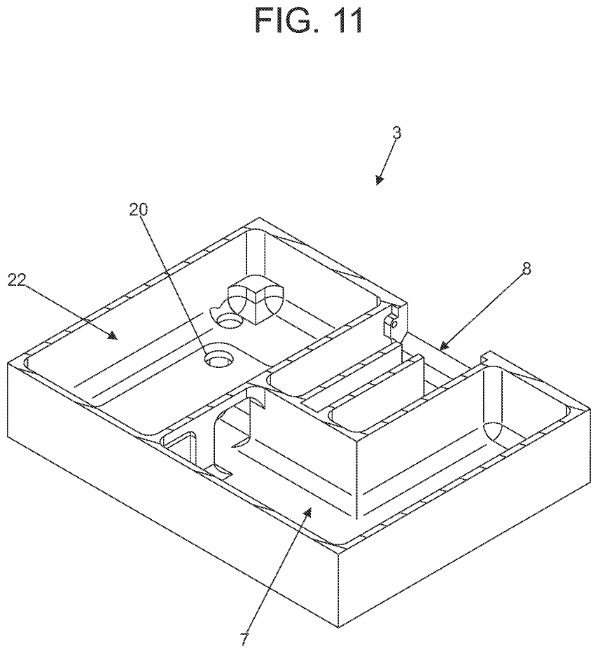

FIG. 12: Detail of the drain of the central reservoir of the arrangement applied in a steam device for cleaning and sanitizing shaving blades.

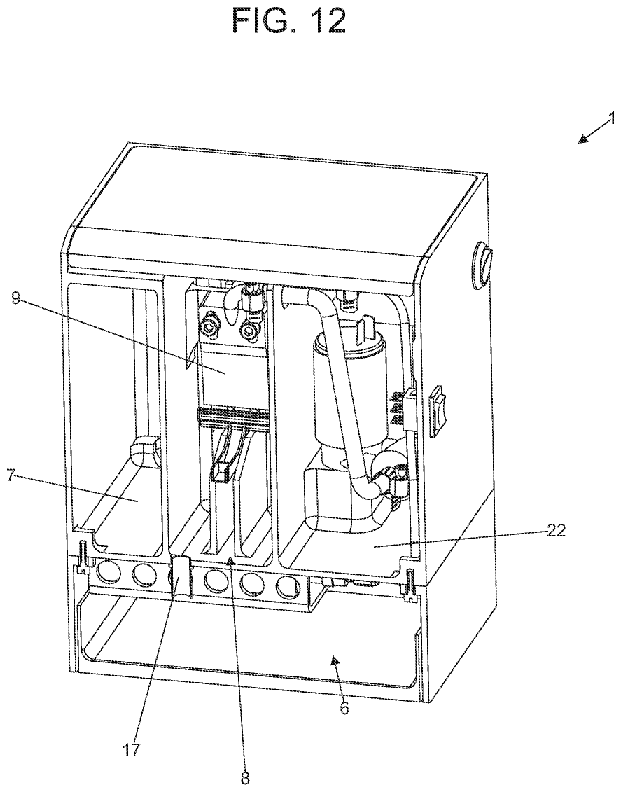

FIG. 13: Detail of the pump drive of the arrangement applied in a steam device for cleaning and sanitizing shaving blades.

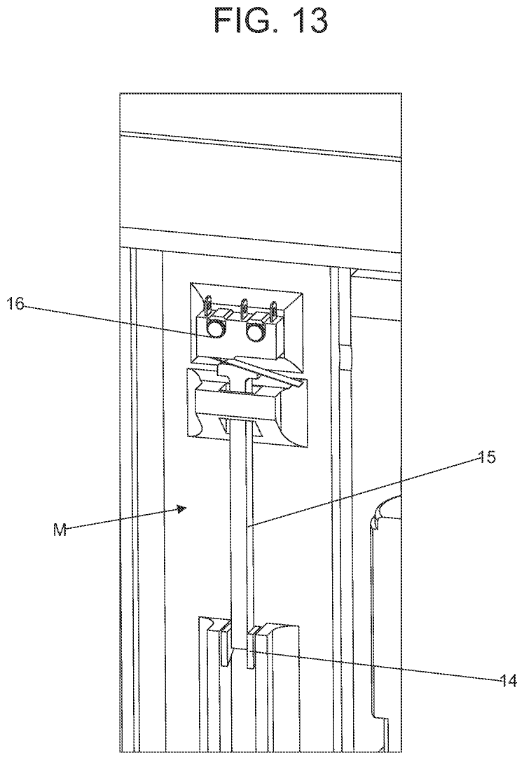

FIG. 14: Detail of the injector nozzle of the arrangement applied in a steam device for cleaning and sanitizing shaving blades, showing the steam outlet.

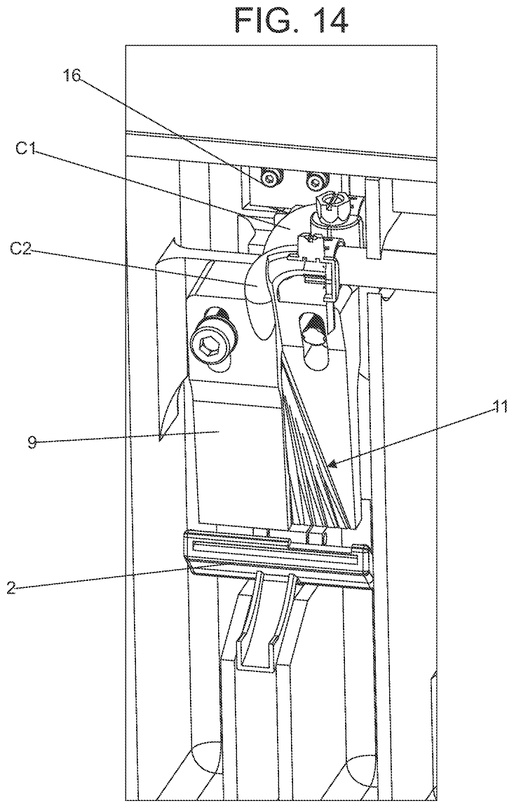

FIG. 15: Detail of the injector nozzle of the arrangement applied in a steam device for cleaning and sanitizing shaving blades, showing the water outlet.

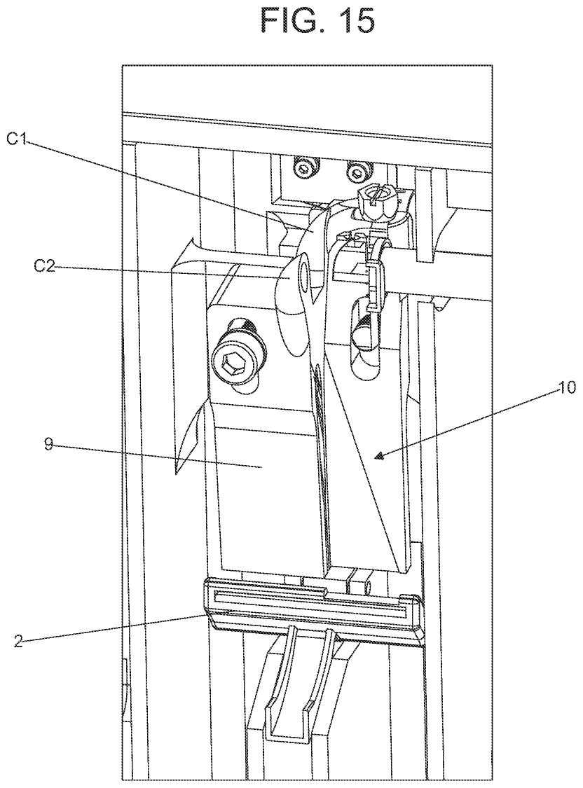

### DETAILED DESCRIPTION OF THE INNOVATION

The ARRANGEMENT APPLIED IN A STEAM DEVICE FOR CLEANING AND SANITIZING RAZOR BLADES, autonomous and compact, formed by a partitioned cabinet composed of main body (3) where an L-shaped compartment (7) acts as water reservoir, utilized to clean and sanitize the blades (2) of razors (13) during use, by the positioning on a support (14) in the central compartment (8) which is part of the drive mechanism (M) of an electric pump (12), which pressurizes the water towards a microboiler (30) making the steam (V) at high pressure and temperature the protagonist of the cleaning.

More particularly, the steam device (1) for cleaning and sanitizing razor blades (2) is based on the use of water steam (V) at high temperature and pressure as protagonist of the cleaning and sanitizing, providing the aseptic cleaning of the blades (2), susceptible to be followed by a flow of pressurized water (A) as complement of the cleaning. The device (1) is assembled in a partitioned cabinet, composed of a main body (3), upper cap (4) for internal access, hatch (5) for inserting the blades (2) and drawer (6) for withdrawal of the residual water, enabling all the operating components to be hermetically encapsulated. Accordingly, the electro-electronic and mechanical parts are preserved. The main body (3) presents an L-shaped seal-tight compartment (7) acting as water reservoir, having sufficient volume to allow the concomitant autonomous use thereof while shaving. A closed central compartment (8), accessed by the tilting front hatch (5), which configures a cleaning chamber where an injection nozzle (9) is located, positioned over the blades (2), formed by two internal channels (C1, C2), wherein channel (C1) receives steam (V) and channel (C2) pressurized water (A). Channel (C1) is finalized at an outlet in the shape of a slit (10) and channel (C2) at an outlet with orifices (11). A drive mechanism (M) of the dual outlet (S1, S2) electric pump (12) is actuated when positioning the head with the blades (2) of the razor (13) on a pivoting support (14) whose rod (15) stops at the end-of-stroke (16) sensor closes contact in the sense of turning said electric pump (12) on. The central compartment (8) has a drain (17) that directs the wash water from the cleaning chamber to a lower compartment (18) to the main body (3) of the cabinet, not before passing through two filters, a coarser mesh screen and a second filter made of washable fabric. In the lower compartment (18) there is a drawer (6) for withdrawing the washing water. The L-shaped compartment (7), water reservoir, communicates by way of internal conduit (20) with the capture (21) of the dual outlet (S1, S2) electric pump (12) installed in a regular prism-shaped compartment (22) which also houses other operating components of the device (1), such as a rechargeable battery (23), a control board (24), a flow valve (25), a low pressure hose (26) interconnected to a high pressure duct (27) that takes pressurized water (A) to the inlet connection (29) of the microboiler (30), installed under the main body (3), from whose outlet (31) derives a second high pressure duct (32) which follows on the channel (C1) of the injection nozzle (9). Other operating components are level sensors, charge indicator of the rechargeable battery (23), timer, drive switch (33) of the microboiler (30) and master switch (34). The control board (24) controls and monitors the charging of the rechargeable battery (23); times the operation of the electric pump (12); controls the temperature of the microboiler (30); monitors the level of the water reservoir; interfaces with the blade positioning switch (2); indicates the level of the rechargeable battery (23) during the operation and charging thereof.

Functionally, the steam device (1) should have the L-shaped compartment (7), accessed by the upper cap (4) fill with drinking water. Afterwards, the master switch (34) is turned on and the electronic system activated, at which time the pre-heating of the microboiler (30) begins, which after some seconds will be at a temperature higher than the boiling point of water. A Led will indicate that the ideal temperature has been reached, signaling that the head containing the blades (2) of the razor (13) can be inserted into the central compartment (8) through the hatch (5), more specifically on the support (14) which is part of the drive mechanism (M) of the electric pump (12). In this condition, upon activating the drive switch (33) of the microboiler (30), the device (1) is ready to operate the steam (V) generated by the microboiler (30) which is already at a high temperature, above the boiling point of water (A), when it contact its chamber, which begins to heat generating high pressure and high temperature steam (V) instantaneously. The high pressure forces the steam (V) through the second high pressure duct (32) which follows on to the channel (C1) of the injection nozzle (9) which directs and further accelerates the steam (V) towards the outlet in the shape of a slit (10) where it meets the blades (2) to be sanitized and in parallel aseptically cleaned. Once this procedure is finalized, after a certain time, the steam is turned off by the timed control system, reversing the power system for pumping pressurized water (A) overflowing from the outlet (S2) towards the channel (C2) of the injection nozzle (9) through an ascending hose (35), complementing the cleaning. After the set time, the working of the device (1) is interrupted, and the user may withdraw the razor through the hatch (5).

1. An ARRANGEMENT APPLIED IN A STEAM DEVICE FOR CLEANING AND SANITIZING RAZOR BLADES, assembled in a cabinet partitioned, working autonomously due to a rechargeable battery (23) which powers the steam device (1) composed of a main body (3), an upper cap (4), a hatch (5) for inserting the razor blades (2) and a drawer (6) for withdrawal of residual water, enabling all operating components to be hermetically encapsulated; a reservoir supplies water to a dual outlet (S1, S2) electric pump (12) with a flow valve (25) which pressurizes the water towards an injection nozzle (9) and from there to the razor blades (2), the residual water, after passing through two filters, being deposited in the drawer (6); the operating components comprising level sensors, a charge indicator of a rechargeable battery (23), a timer, a drive switch (33) of a microboiler (30) and a master switch (34) characterized in that the main body (3) presents an L-shaped seal-tight compartment (7) acting as a water reservoir; a central compartment (8) with a drain (17), accessed by a front tilting hatch (5); a regular prism-shaped compartment (22) housing the operating components and a lower compartment (18) for the drawer (6); the dual outlet (S1, S2) electric pump (12) by means of a low pressure hose (26) interconnected to a high pressure duct (27) takes water (A), collected in a internal conduit (20), pressurized up to an inlet connection (29) of the microboiler (30), installed under the main body (3), from whose outlet (31) derives a second high pressure duct (32) that follows on to a channel (C1) of the injection nozzle (9), in the central chamber (8), which directs and further accelerates steam (V), protagonist of the cleaning, towards the outlet in the shape of a slit (10) to meet the razor blades (2) to be sanitized and in parallel aseptically cleaned; the injection nozzle (9) has a channel (C2) for pressurized water (A) without an outlet in the shape of orifices (11); the pressurized water (A) comes from the outlet (S2) and follows on through an ascending hose (25) up to the channel (C2); after a certain time, a control board (24) ceases the steam (V) and reverses the system for pressurized water (A) to complement the cleaning; in the central compartment (8) there is a drive mechanism (M) of the dual outlet (S1, S2) electric pump (12) which is actuated when positioning the head with the blades (2) of the razor (13) on a pivoting support (14) whose rod (15) when stopping at the end-of-stroke sensor (16); the control board (24) controls and monitors the charging of the rechargeable battery (23); times an operation of the electric pump (12); controls the temperature of the microboiler (30); monitors the level of the water reservoir; interfaces with the blade positioning switch (2); indicates the level of the rechargeable battery (23) during the operation and charging thereof.
